(19) United States
(12) Patent Application Publication
(10) Pub. No.: US 2013/0084338 A1
(43) Pub. Date: Apr. 4, 2013

(54) Method For Producing Solid Pigment-Containing Film Coating Compositions In The Form Of Granules Based On Enteric Film Formers For Pharmaceutical Dosage Forms

(71) Applicant: BASF SE, Ludwigshafen (DE)

(72) Inventors: Angelika Maschke, Mannheim (DE); Karl Kolter, Limburgerhof (DE); Kathrin Meyer-Böhm, Feucht (DE); Thorsten Schmeller, Wachenheim (DE)

(73) Assignee: BASF SE, Ludwigshafen (DE)

(21) Appl. No.: 13/632,335

(22) Filed: Oct. 1, 2012

Related U.S. Application Data
(60) Provisional application No. 61/541,111, filed on Sep. 30, 2011.

(51) Int. Cl. B05D 1/02; A61K 9/50

(52) U.S. Cl. 424/490; 427/2.23

(57) ABSTRACT

Method for producing pigment-containing granules for pharmaceutical applications based on film-forming enteric polymers, wherein the production of the granules takes place by a spraying process in which the enteric polymeric film formers are introduced as initial charge in a fluidized bed, and an aqueous pigment suspension, which comprises a plasticizer that is solid at 20° C., is sprayed onto the fluidized bed.

The present invention relates to a novel method for producing solid pigment-containing film coating compositions in the form of granules based on enteric Him formers for the coating of pharmaceutical dosage forms. Furthermore, the invention relates to the corresponding granules and the use thereof for producing enteric coated drug forms.

Enteric film formers and pigment-containing granules obtained therefrom for producing pharmaceutical film coatings are known per se.

According to the prior art, “enteric” is the term used to refer to polymers which are insoluble in an acidic environment and, above a pH in the range from 4.5 to 6.5, become soluble as a result of dissociation of the carboxylic acid group, with salt formation, such that, at 37 +/−0.5° C. from an active-ingredient-containing core coated with the polymer, in 750 ml of 0.1 N HCl after two hours, only at most 10% by weight of the amount of active ingredient are released, but in phosphate buffer, at pH 6.8, the release of at least 90% by weight takes place within 45 minutes. The apparatus used is described in the pharmacopeia: USF: Dissolution <711>, Paddle apparatus; Ph. Eur.: Dissolution test for solid dosage forms Ch. 2.9.3). This definition and method of determination are also applicable for the granules according to the invention.

Usually, to produce enteric film coatings, the individual constituents are weighed in separately and processed. Processing requires a homogenization step so that the pigments are deagglomerated and comminuted to the required fineness. This step is complex and poorly reproducible since it is heavily dependent on the batch size, the equipment and the processing conditions. Often, foam formation also arises, again can lead to the partial agglomeration of the pigments and which requires a certain waiting time before further processing. In addition, the plasticizer has to be incorporated; this can lead to coagulation of the polymer dispersion and, moreover, this step as well requires a certain time.

Overall, the production of ready-to-spray preparations with the many weighing-in steps, the homogenization using a high-shear stirrer and the stepwise production of part preparations, which are only combined later, is very time-intensive, cleaning-intensive and labor-intensive. Added to this is the fact that polymer dispersions coagulate very easily in hot and cold countries and thus become unusable.

These disadvantages could be avoided by dry powders which comprise all constituents. They should be more storage-stable, quick and easy to disperse and reproducible in use.

Enteric polymers are very brittle in the dry state under certain circumstances, which can lead to cracking problems in the coatings. However, such cracks in the coatings cannot be tolerated for reasons of drug safety. For this reason, plasticizers are usually added.

However, the pigment-containing granules should also be storage-stable, not stick together and in particular not separate because this has an adverse effect on the dosability during further processing.

EP-A 152038 describes the production of pigmented pharmaceutical coating compositions based on enteric polymers, where the coating composition takes place by mixing an aqueous dispersion of the enteric polymer with a pigment suspension.

DE-A 3127237 describes the production of pigmented coating compositions based on (meth)acrylic acid polymers, where an aqueous dispersion of the polymers is mixed with polyethylene glycol, talc and titanium dioxide. Similar aqueous coating compositions are also described in U.S. Pat. No. 3,935,326.

EP-A 342 106 describes PEG 6000-comprising aqueous coating compositions based on (meth)acrylic acid copolymers such as Eudragit L30D and Eudragit NE 30D, which are obtained using the aqueous dispersions of the copolymers.

EP 194 838 describes aqueous coating compositions which are obtained by adding microcrystalline cellulose, PEG 6000 and water to an aqueous Eudragit L30D-55 dispersion and than mixing in a high-speed mixer.

WO 00/18375, as well as WO 03/070224, WO 2008/002808 and WO 2008/080774 describe the use of water-soluble polyether-polyvinyl alcohol copolymers in pigmented pharmaceutical coating compositions.

WO 2007/006353 describes the use of acidic (meth)acrylate copolymers in partially neutralized form for producing coating compositions, where the coating compositions can in principle also comprise relatively high molecular weight polyethylene glycols as plasticizers. Specifically, however, only aqueous coating compositions which comprise triacetin as plasticizer are described.

WO 2009/004649 describes the production of an enteric coating composition where art aqueous dispersion of a methacrylic acid copolymer is admixed with PEG 8000, talc, titanium dioxide, Polysorbate 80 and sodium hydroxide.

U.S. Pat. No. 4,566,652 describes the production of enteric coating compositions, where a dry mixture of polyvinyl acetate phthalate, PEG 3380, aluminum colored lakes and further constituents is mixed in a jacketed mixer and is then ground in a hammer mill.

In U.S. Pat. No. 4,543,370 dry pulverulent pharmaceutical coating compositions are obtained by mixing a dry mixture of a polymer with titanium dioxide, PEG 400 and further constituents in a mixer and then comminuting it by grinding.

WO 2010/182204 describes the production of pulverulent coating compositions of polyvinyl alcohol, methacrylic acid ethyl acrylate copolymer, sodium bicarbonate, talc, titanium dioxide and solid polyethylene glycol by dry mixing the components in a mixer.

U.S. Pat. No. 6,420,473 describes solid pulverulent coating compositions which are obtained by, in a first step, a mixing of methacrylic acid-ethyl acrylate copolymer powder with sodium bicarbonate and further solid constituents such as titanium dioxide, talc and further auxiliaries in a mixer faking place, and the solid mixture obtained is then mixed with the plasticizer triethyl citrate. The dry powder obtained in this way is then redispersed. In water in a vortex mixer. However, such dry mixtures are not storage-stable to a satisfactory degree.

Coating compositions in the form of dry powders known hitherto often have problems with filming and homogeneity of the produced films. Consequently, the resistance to gastric fluid and thus the resistance to protons is unsatisfactory. Moreover, the dust fractions are often very high.

On the other hand, however, there is also the problem of often inadequate storage stability and of separation, and also of lack of drug safety. This also gives rise to problems with redispersiblity which is then not ensured to an adequate extent. Also when mixing with further color-imparting agents to expand the color space, separation phenomena are a major problem.

It was an object of the present invention to provide dry pulverulent free-flowing granules with low dust fractions for pharmaceutical enteric coating compositions which do not have the disadvantages of the prior art.

Accordingly, a method for producing pigment-containing granules for pharmaceutical applications based on film-forming enteric polymers has been found which is characterized in that the production of the granules takes place by a spraying process in which the enteric polymeric film former is introduced as initial charge in a fluidized bed, and an aqueous pigment suspension, which comprises a plasticizer that is solid at 20° C., is sprayed onto the fluidized bed.

In principle, enteric film formers that are suitable according to the invention are all acid group-containing polymers with a corresponding solubility behavior, the enteric dissolution behavior of which is controlled through the presence of the acid groups. According to the invention, enteric polymers is the term used to refer to those polymers which, under standard conditions (room temperature 20 to 25° C., atmospheric pressure) at pH values of less than 4.5, have a solubility in water of less than 1% by weight, and at pH values above 6.5 have a solubility in water of more than 10% by weight and up to 70% by weight.

Thus, according to the invention, as well as (meth)acrylates, cellulose derivatives such as, for example, hydroxypropylmethylcellulose phthalates, cellulose acetate phthalates or hydroxypropylmethylcellulose acetate succinates, are also suitable.

According to a preferred embodiment of the invention, the enteric film formers used are methacrylic acid-acrylate copolymer. According to the invention, methacrylic acid acrylates is the term used generally to refer to copolymers of methacrylic acid which display enteric solubility.

Thus, enteric film formers which can be used are copolymers of methacrylic acid and ethyl acrylate in the monomer ratio of 1:1. Also of suitability are copolymers of methacrylic acid and methyl methacrylate in the molar ratio 1:1 or copolymers of methacrylic sold and methyl methacrylate in the molar ratio 1:2.

These polymers are monographed in the pharmacopeia and are listed, for example according to US Pharmacopeia, as methacrylic acid copolymers of types A, B, C. Such methacrylic acid-acrylate polymers are commercially available.

Particular preference is given to copolymers of methacrylic acid and ethyl acrylate in the monomer ratio 1:1 (PMEA 1:1) with average molecular weights M_{w} in the range from 250000-280000. The molecular weight distribution was determined by gel permeation chromatography compared to a polymethyl methacrylate (PUMA) standard.

The copolymers can also be used in partially neutralized form. Of suitability is thus, for example, Kollicoat® MAE 100P commercially available pulverulent PMEA 1:1 copolymer which has been partially neutralized with sodium hydroxide, such that in the region of 6 mol % of the acid groups are present in neutralised form. This pulverulent polymer has particle sizes of 155 μm ((d(4,3) volume average) determined by light diffraction. The measurement can take place using a Malvern Mastersizer.

According to one embodiment of the invention, which is based on the use of enteric polymers which have not been partially neutralized beforehand, for example Eudragit® L100 grades, an alkali addition takes place during the agglomeration in order to ensure adequate redisperability of the granules. The alkali addition can be added here as powder to the initial charge consisting of solid enteric polymer, or it can be dissolved in the spraying solution. In a particular embodiment, the initial charge consisting of solid enteric polymer is sprayed firstly with an aqueous solution of the alkali addition, and only then are the other components sprayed on. Suitable alkali additions are: alkali metal or alkaline earth metal hydroxides, alkali metal or alkaline earth metal carbonates, alkali metal or alkaline earth metal hydrogencarbonates, basic salts of physiologically compatible acids such as e.g. trisodium phosphate, trisodium citrate.

The enteric polymers can be used in amounts such that 50 to 93% by weight, based on the solids content, preferably 70 to 90% by weight, of enteric polymer are present in the finished granular film coating composition.

According to the invention, the enteric polymers are introduced as initial charge in the form of powders in a fluidized bed. The pulverulent polymers used can have particle sizes in the range from 50 μm to 800 μm.

According to the invention, pigments which can be used in the granular film coating composition are inorganic pigments or organic pigments or mixtures thereof. Preference is given to using white pigments as pigments. Of particular suitability as pigments are titanium dioxide, zinc oxide, kaolin, calcium or magnesium phosphates, silicon dioxide, bentonite or silicates. Talc is likewise suitable in principle as pigment, but, in connection with this invention, is referred to as antisticking agent.

The pigments can be added in amounts such that 2 to 30% by weight, preferably 2 to 18% by weight, based on the solids content, of pigment are present in the finished film coating composition.

According to one embodiment of the invention, plasticizers are added to the aqueous pigment dispersion.

According to a preferred embodiment, the granular film coating composition already comprises the plasticizers.

Suitable plasticizers are in principle all plasticizers customary in the field of pharmaceutical formulations which are solid at 20° C. and atmospheric pressure. Additionally, it is also possible to add liquid plasticizers in a small amount.

Preference is given to using plasticizers which nave melting points of from 30 to 100° C.

For example, plasticizers which can be used are polyethylene glycols with molecular weights of from more than 600 g/mol to 15000 g/mol, preferably 1500 to 8000 g/mol

Furthermore, plasticizers which can be used are polyethylene glycol-polyvinyl alcohol copolymers, for example the commercially available Kollicoat® IR, BASF SE, a graft copolymer of PEG 6000 and polyvinyl alcohol units in the molar ratio 25:75 with an average molecular weight in the region of 45000 g/mol.

Furthermore of suitability are also poloxamers, i.e., polyethylene oxide-polypropylene oxide block copolymers, for example Poloxamer 407, commercially Lutrol®F127 (average relative molar mass 3340-14 600) or Poloxamer 168, commercially Lutrol® F66 (average relative molar mass 7880-9510).

Particular preference is given to using polyethylene glycols (PEG) with molecular weights of from 1500 to 6000 g/mol. In particular PEG 8000.

The plasticizers can be added in amounts such that 5 to 25% by weight preferably 7 to 15% by weight, based on the solids content of the granules, of plasticizer are present in the finished film coating composition.

According to one embodiment of the invention, the aqueous pigment dispersions additionally comprise antisticking agents. If antisticking agents are used, then these can be added in amounts of from 1 to 20, preferably 5 to 20% by weight, based on the solids content of the granules.

Suitable antisticking agents are, for example, talc, silicates, glycerol monostearate, stearic acid, stearyl alcohol.

Preference is given to using talc as antisticking agent. Within the context of this invention, talc is treated as an antisticking agent with regard to the quantitative data.

The feed materials pigments, plasticizers and antisticking agents are used in total in amounts such that the aqueous pigment dispersions have contents of from 5 to 40% by weight.

The resulting granules can have the following composition, the quantitative data referring in principle to the total weight of the granules (=100% by weight):
(i) 50 to 93% by weight of enteric polymers
(ii) 2 to 30% by weight of pigments
(iii) 5 to 25% by weight of plasticizers
(iv) 0 to 30% by weight of antisticking agents
(v) 0 to 10% by weight of further auxiliaries,

Preferred compositions are:
(vi) 70 to 90% by weight of enteric polymers
(vii) 2 to 18% by weight of pigments
(viii) 7 to 15% by weight of plasticizers
(ix) 1 to 20% by weight of antisticking agents
(x) 0 to 5% by weight of further auxiliaries.

Particularly preferred compositions are:
(xi) 75 to 80% by weight of enteric polymers
(xii) 2 to 8% by weight of pigments
(xiii) 10 to 15% by weight of plasticizers
(xiv) 7 to 12% by weight of antisticking agents
(xv) 0 to 2% by weight of further auxiliaries.

According to a very particularly preferred embodiment, the granules comprise 75 to 80% by weight of a partially neutralised copolymer of methacrylic acid and ethyl acrylate in the monomer ratio 1:1, 2 to 8% by weight of pigments, 10 to 15% by weight of a polyethylene glycol with molecular weights in the range from 1500 to 8000 daltons as plasticizer and 7 to 12% by weight of talc as antisticking agent.

According to the invention, the production method according to the invention takes place in such a way that an aqueous pigment dispersion comprising a plasticizer that is solid at 20° C. is sprayed onto a fluidized bed of the pulverulent enteric polymeric film former.

As already mentioned, the production takes place by a spray-agglomeration process.

The spray-agglomeration process can he earned out discontinuously, semicontinuously or continuously.

According to one embodiment of the invention, the process is carried out as a discontinuous process, in which case a defined amount of the pulverulent polymeric film former is introduced as initial charge.

The amount of polymeric film former introduced as initial charge in the fluidized bed is governed by the size of the spraying device.

The particle size of the enteric polymer introduced as initial charge in the fluidized bed is in the range from 50 μm to 800 μm (average particle diameter, d(4,3) value, weighted volume average), preferably 100 to 400 μm.

The entry air temperature in the spraying process is less than 100° C., preferably less than 90° C., particularly preferably less than 60° C. The entry air temperature chosen in a specific case is governed by the desired product temperature in the fluidised bed. In particular, the entry air temperature is 20 to 50° C.

The exit air humidity during the spraying process is usually in the range from 20 to 50% relative humidity.

The product temperature in the fluidised bed can be in the range from 15 to less than 60° C., preferably in the range from 20 to 50° C., particularly preferably 25 to 45° C.

The spraying pressure of the aqueous pigment dispersion can be in the range from 0.05 to 0.5 MPa, preferably 0.01 to 0.4 MPa, particularly preferably 0.01 to 0.3 MPa.

According to one embodiment according to the invention, the spraying process takes place at entry air temperatures of from 20 to 50° C. and a product temperature of from 20 to 50° C.

According to a further embodiment of the invention, following the spraying process using the aqueous pigment-containing spraying suspension, a further process step can be carried out in which an aqueous dispersion of the enteric polymer is sprayed onto the preformed granules. In this embodiment, 2 to 10, preferably 5 to 7% by weight of the total amount of enteric polymer in the granules are introduced by means of this downstream spraying stage. The aqueous dispersions of the enteric polymer can be adjusted to solids content of from 10 to 30% by weight.

The aqueous dispersion during processing is not warmer than 40° C. and, if required, is cooled prior to the spraying. Preference is given to cooling to 20-25° C.

For all embodiments of the process according to the invention, the particle sizes of the resulting granules are in the range from 200 to 1000 μm (d(4,3) value, weighted volume average, determined by means of light diffraction), preferably in the range from 250 to 800 μm.

According to the invention, the granules obtained in this way are suitable for producing coating compositions for the production of enterically coated drug forms. Such drug forms, which are also referred to in Anglo-American speak as “delayed release dosage forms” release less than 10% of the active ingredient within 120 min after oral administration. The release is determined in accordance with USP <711>: 120 min gastric fluid, then rebuffering (0.08 M HCL as gastric fluid, after 2 h rebuffering to pH 6.8 with phosphate buffer).

To produce the aqueous coating compositions for the coated drug forms, the granules according to the invention, which, on account of their titanium oxide content, have a white or whitish appearance, are also admixed with further pigments or pigment preparations to achieve colored coatings. In particular, the coating compositions according to the invention are suitable for mixing with commercially available colored ready-to-use coating compositions cased on polyether-polyvinyl alcohol graft copolymers, which are available under the trade name Kollicoat® IR Coating Systems from BASF SE. These are colored ready-to-use coating compositions of 58.1% by weight of Kollicoat IR (graft polymer polyethylene glycol-polyvinyl alcohol in the quantitative ratio 25:75, average molecular weight 45000), 6.4% Kollidon® VA 64, 8.4% TiO₂, 1.9% sodium lauryl sulfate, 9.9% colored pigment. Although these colored ready coating compositions are intended for the application of instant release dosage forms, they can he mixed in suitable amounts with the granules according to the invention without the resulting colored coatings losing their resistance to gastric fluid. Preferably, the colored ready mixtures are mixed with the granules according to the invention in amounts such that the resulting colored coating composition comprises a fraction from 70 to 98% by weight, preferably from 80 to 95% by weight, based on the solids content, of granules according to the invention.

Additionally, further plasticizers such as, for example, triacetic, triethyl citrate, diethyl sebacate, acetyl triethylcitrate, propylene glycol, polyethylene glycol 300-600, can be added to the redispersed granular coating compositions.

Further auxiliaries which can be used are:

viscosity-increasing polymers such as e.g. xanthan, carrageenars, alginate, pectin, hydroxypropylmethylcellulose, surfactants such as e.g. sodium lauryl sulfate, Cremophor® RH 40, Polysorbate 80, Eumulgin®2 PH, luster agents, dispersion auxiliaries, protective colloids, antioxidants (e.g. butylhydroxytoluene, butylhydroxyanisole, N-acetylcysteine), lyophilizing agents.

To produce an aqueous spraying suspension, the granules according to the invention are redispersed in water with stirring using a simple stirrer, e.g. a paddle stirrer. This redispersion is usually completed after 10 minutes and the spraying suspension can he used. On account of the particular granular structure, dumping cannot arise. The granules according to the invention can be added in a large amount in a short time.

Moreover, if is of particular advantage that no foam is formed during the redispersion.

The coating compositions can be used for coating tablets, hard capsules, soft capsules, pellets, granules, crystals, extrudates.

Surprisingly, granules obtained by the method according to the invention can be processed to give aqueous coating compositions, the properties of which avoid the problems of the prior art. In this connection, the very good redispersibility was particularly surprising.

The very good flow properties offer major advantages during processing, particularly on account of the good dosability. This is of fundamental importance for reasons of drug safety.

Emphasis is to be placed particularly on the freedom from dust of the granules according to the invention, which is important particularly during processing under BMP conditions (Good Manufacturing Practise) on account of the risk of cross contamination. Furthermore, it leads to time savings on account of complex cleaning procedures of equipment and surroundings contaminated with dust being avoided. Moreover, personnel do not breathe in dust.

It was also unexpected that despite processing in the presence of water above the minimum film-forming temperature of the mixture of plasticizer and enteric polymer, no filming of the polymer particles takes place. Under such circumstances, the person skilled in the art would have expected filming and thus agglutination of the agglomerates. Surprisingly, despite processing in the presence of water, the plasticizer also does not diffuse into the polymer particles.

The storage stability of the dry mixture is also excellent. In contrast to mixtures known hitherto, separation of the compounds and also clumping do not arise, even upon prolonged storage and transportation. Separations, as often arise in the case of simple powder mixtures, would be particularly critical because, as a result, the resistance to gastric fluid of all presentation forms would no longer be present, and corresponding drugs would become ineffective. As a result of vibrations e.g. during transportation, in the case of simple powder mixtures, the light constituents collect at the top, and the heavy ones at the bottom. There is often also a separation according to size and structure of the particles. Consequently, the polymer can be separated from the pigment and from the plasticizer, with the stated fatal consequences. The granules according to the invention are completely stable against separation.

### EXAMPLES

The determination of average particle sizes (d 4,3), weighted volume average, was carried out by means of light diffraction using a Malvern Mastersizer 2000.

All release tests in accordance with USP <711> were carried out using a paddle apparatus at 37+/−0.5° C.

The agglomerates were produced by spraying processes in the fluidized bed.

General procedure for producing the spraying suspension:

The pigments TiO₂ and talc were dispersed in water and then the plasticizer was dissolved in this pigment suspension. The spraying suspension was adjusted to solids contents of 35 or 40% solids content, the dynamic viscosity was in the range 25-35 mPas, determined using a rotary viscometer (Haake Rheowin 322) at 23° C.

As enteric film former, Kollicoat MAE 100 P, BASF SE, was introduced as initial charge in the fluidized bed (PMEA 1:1 copolymer which has been partially neutralised with sodium hydroxide such that in the region of 6 mol % of the acid groups are present in neutralized form, Mw 250000). The polymer powders comprise, based on the total solids content, 0.7% sodium lauryl sulfate and 2.3% by weight Polysorbate 80. The average particle size (weighted volume average (d 4,3), light diffraction) was 144 μm.

### Examples 1 to 5

### Composition of the Resulting Agglomerates: Numerical Data in % by Weight

| | Example 1 | Example 2 | Example 3 | Example 4 | Example 5 |
|---|---|---|---|---|---|
| Kollicoat | 75.7 | 79.1 | 79.1 | 88.18 | 88.18 |
| MAE 100P | | | | | |
| PEG 6000 | 11.3 | 7.9 | | 8.82 | |
| PEG 1500 | | | 7.9 | | 8.82 |
| Titanium | 3 | 10 | 10 | | |
| dioxide | | | | | |
| Talc | 10 | 3 | 3 | 3 | 3 |

### Example 1

The resulting granules have particle sizes in the region of 329 μm (weighted volume average d(4,3), light diffraction).

The agglomeration was carried out in an instrument from Glatt (Glatt GPCG 3). The charging of the fluidized bed, the spraying rates and the entry air temperatures were varied as stated below.

Settings of Glatt GPCG 3

| | |
|---|---|
| Bed initial charge | 1028 g or 984 g |
| (Kollicoat ® MAE 100P) | |
| Container size | 10 l |
| Die | 1 mm |
| Spraying rate | varied from 17 g/min or 28 g/min |
| Entry air temperature | 25° C. in the case of PEG 1500 |
| | 50° C. in the case of PEG 6000 |
| Spraying pressure | 0.15 MPa |
| Product temperature | kept in the range 25-30° C. |
| Exit air humidity | kept in the range 40-50% |
| Volume stream | 90-110 m³/h |

Preparation of caffeine tablet cores:

### Composition of the Tabletting Mixture (Data in % by Weight)

| | |
|---|---|
| Caffeine gran 02/05 | 15.5% |
| Ludipress*) LCE | 74.0% |
| Kollidon ® CL-F | 5.0% |
| Kollidon ® VA 64 Fine | 5.0% |
| Mg stearate | 0.5% |

*)physical mixture of 93% lactose, 3.5% Kollidon ® 30 (PVP K30) and 3.5% Koillidon ® CL (crospovidon)

Tablets were pressed from this tabletting mixture.

Setting on the Korsch rotary XL 100 tablet press

| | |
|---|---|
| Tablet format: | 9 mm, convex with engraving |
| Tablet weight: | 350 mg |
| Pressing force: | 10 kN |
| Tabletting speed: | 60 rpm/min |
| Agitator blade filling shoe: | 15 rpm/min |
| Filling curve | 8-mm |

The tablet cores produced in this way were then provided with enteric film coatings. To produce the film coating compositions, aqueous spraying suspensions were produced with the agglomerates obtained according to Examples 1 to 5, where the agglomerates were stirred at room temperature for 120 min. The concentration was selected such that spraying suspensions with solids contents of 20% by weight were obtained. To produce the coated tablets, a laboratory tablet coater of the type Manesty XL Lab 01 was used.

| | |
|---|---|
| Process parameter | Manesty XL Lab 01 |
| Batch size (tablet amount) | 5 kg |
| Application amount | 6.8 + 10 mg/cm² |
| Drum size | 7 l tablet volume (medium drum) |
| Drum speed | 14 rpm |
| Internal pressure | 50 |
| Entry air stream | 450 m³/h |
| Entry air temperature | 55° C. |
| Spraying rate | 25% (23-25 g/min) |
| Atomization air | 0.28 MPa |
| Spraying width | 2.8 bar |
| Temperature of tablets | 33-34° C. |

In order to take into consideration the influence of mechanical stress during the packaging of the coated tablets, the coated tablets were subjected to a mechanical stress test by stressing them in the friabilator for 4 minutes. Here, the conditions and the apparatus ware used which are described according to PharmEur. 7.0, chapter 2.9.7 for the friability determinations on noncoated tablets. Brittle films would be damaged thereby and would exhibit poorer resistance to gastric fluid.

The release of the tablets was then determined in accordance with the method USP <711> “Delayed Release Tablets”:

120 min gastric fluid, then rebuffering

0.08M HCl as gastric fluid, after 2 h rebuffering to pH 6.8

All tablets showed resistance to gastric fluid, which means that less than 10% active ingredient were released in the first 120 min.

### Example 6

### Lutrol® F 80 (Poloxamer 188) as Plasticizer

| Constituent | Solids | Mixture |
|---|---|---|
| Kollicoat MAE 100P | 88.5% | 500 g |
| Lutrol F 68 | 8.85% | 150 g |
| Titanium dioxide | 2.65% | 45 g |
| Water | — | 460 g |
| Concentration of the spraying solution | | 30% |
| Sprayed-on amount per 500 g of | | 218 g |
| Kollicoat MAE 100 P | | |

To produce the spraying suspension, firstly 60% of the water were weighed out, then the plasticizer was added and dissolved. The remaining water was then weighed out and TiO2 was dispersed therein, and this suspension was added to the plasticizer solution. Granulation was carried out under the conditions given below.

| | |
|---|---|
| Process settings | Glatt GPCG3 |
| Bed initial charge | 500 g |
| (Kollicoat ® MAE 100P) | |
| Container size | 5 l |
| Die | 0.8 mm |
| Spraying rate | 10-11 g/min |
| Entry air temperature | 35° C. |
| Spraying pressure | 0.15 MPa |
| Product temperature | held between 25-30° C. |
| Exit air humidity | held between 40-50% |
| Volume stream | 90-110 m³/h |

A 20% strength by weight spraying suspension was prepared from the granules obtained in this way by adding the corresponding amount of granules to demineralized water and stirring for 90 min at room temperature.

| | |
|---|---|
| Process settings | Manesty XL Lab 01 |
| Batch size (tablet amount) | 5 kg caffeine cores |
| Application amount varies | 3/4/6/8 or 10 mg/cm² |
| Drum size | 7 l tablet volume (medium drum) |
| Drum speed | 14 rpm |
| Internal pressure | 50 |
| Entry air stream | 450 m³/h |
| Entry air temperature | 55° C. |
| Spraying rate | 25% (20 g/min) |
| Atomization air | 0.28 MPa |
| Spraying width | 0.28 MPa |
| Temperature of tablets | 31-34° C. |

Stressing the tablets:

The tablets were stressed for 4 min in the friabilator in order to assess the influence of the stress on the resistance to gastric fluid.

Active Ingredient release (USP <711>)

120 min gastric fluid, then rebuffering

0.08M HCl as gastric fluid, after 2 h rebuffering to pH 6.8

The requirement for resistance to gastric fluid was satisfied for stressed tablets even in the case of low application amounts of 3 mg/cm².

### Example 7

### Kollicoat IR as Plasticizer

Formulation: data in % by weight

| | | |
|---|---|---|
| Kollicoat MAE 100P | 87.80% | 82.09% |
| Kollicoat IR | 8.78% | 14.49% |
| Titanium dioxide | 3.42% | 3.42% |

The preparation was carried out analogously to Examples 1 to 5

### Example 8

The preparation was carried out analogously to Example 1, although the amount of Kollicoat MAE in the fluidized bed was reduced by 5% by weight, based on the total fraction of Kollicoat MAE. This fraction was dispersed in wafer and sprayed onto the finished granules in the form of a 20% strength by weight spraying suspension.

### Example 9

### Preparation of Colored Coating Compositions

Granules obtained according to Example 1 were mixed as stated below with a colored ready coating composition+) in the mixing ratios granules to Kollicoat IR CS of 80:20 and 90:10 and with triacetin as plasticizer, and sprayed onto the tablet cores described above.

+) Kollicoat® IR Coating Systems from BASF SE (Kollicoat IR CS): colored ready-to-use coating composition comprising 58.1% by weight of Kollicoat IR (graft polymer polyethylene glycol-polyvinyl alcohol in the quantitative ratio 25:75, average molecular weight 45000) 8.4% Kollidon® VA 64, 8.4% TiO2, 1.9% sodium lauryl sulfate, 9.9% colored pigment.

### Percentages in % by Weight Based on the Total Amount of Coating Compositions and Plasticizers

| | 80:20 | 90:10 |
|---|---|---|
| Granules as in Ex. 1 | 74.1% | 82.6% |
| Kollicoat IR CS | 18.5% | 9.2% |
| Triacetin | 7.4% | 8.2% |

The aqueous spraying suspension was produced such that the solids content was 20% by weight. To produce the spraying suspension, firstly the water was weighed out, than the plasticizer was added and dissolved. The granules according to Example 1 were then added and stirred using a paddle stirrer for 4 hours until a homogeneous dispersion was achieved. The respective amounts of Kollicoat IR Brilliant Blue or Yellow were then added and stirred overnight under room conditions. The tablet cores were coated with the finished colored spraying suspension as stated below.

| | |
|---|---|
| Settings | Manesty XL Lab 01 |
| Tablet amount | 5 kg |
| Application amount | 10 mg/cm² |
| Drum size | 7 l tablet volume (medium drum) |
| Drum speed | 14 rpm |
| Internal pressure | 50 |
| Entry air stream | 450 m³/h |
| Entry air temperature | 55° C. |
| Spraying rate | 25% (20 g/min) |
| Atomization air | 0.28 MPa |
| Spraying width | 0.28 MPa |
| Temperature of tablets | 31-34° C. |

Unstressed tablets and tablets stressed in the friabilator for 4 min satisfied the requirement “resistance to gastric fluid”.

### Examples 10 to 12

The preparation was carried out analogously to Examples 1 to 5.

### Examples 13, 14

The preparation was carried out analogously to Example 8.

The process parameters and particle sizes are listed in the table below.

| | | Process parameter | | | | | | Particle |
|---|---|---|---|---|---|---|---|---|
| | | Entry air | Product | Exit air | Exit air | Volume | Spraying | sizes |
| Example | Composition | temp. | temp. | temp. | humidity | stream | rate | D (4,3) |
| 10 | 75% Kollicoat MAE 100P | 50° C. | 34° C. | 34° C. | 43-48% | 149 m³/h | 21-25 g/min | 263 μm |
| | 10% PEG 6000 | | | | | | | |
| | 12% talc | | | | | | | |
| | 3% TiO2 | | | | | | | |
| 11 | 75% Kollicoat MAE 100P | 50° C. | 35° C. | 34° C. | 45-49% | 160 m³/h | 21-26 g/min | 413 μm |
| | 15% PEG 6000 | | | | | | | |
| | 7% talc | | | | | | | |
| | 3% TiO2 | | | | | | | |
| 12 | 75.7% Kollicoat MAE | 50° C. | 34° C. | 34° C. | 42-51% | 162 m³/h | 21-25 g/min | 396 μm |
| | 100P 11.3% PEG 6000 | | | | | | | |
| | 10% talc | | | | | | | |
| | 3% TiO2 | | | | | | | |
| 13 | 74.1% Kollicoat MAE | 35° C. | 23° C. | 23° C. | 49% | 85 m³/h | 11 g/min | 376 μm |
| | 100P 7.9% PEG 6000 | | | | | | | |
| | 10% talc | | | | | | | |
| | 3% TiO2 | | | | | | | |
| | 5% Kollicoat MAE 100P | | | | | | | |
| | as coating | | | | | | | |
| 14 | 83.18% Kollicoat | 35° C. | 24° C. | 24° C. | 47% | 81 m³/h | 11 g/min | 409 μm |
| | MAE100P | | | | | | | |
| | 8.82% PEG 6000 | | | | | | | |
| | 3% TiO2 | | | | | | | |
| | 5% Kollicoat MAE 100P | | | | | | | |
| | as coating | | | | | | | |

1. A method for producing pigment-containing granules for pharmaceutical applications based on film-forming enteric polymers, wherein the production of the granules takes place by a spraying process in which an enteric polymeric film former is introduced as initial charge in a fluidized bed, and an aqueous pigment suspension which comprises a pigment and a plasticizer, the plasticizer being solid at 20° C. is sprayed onto the fluidized bed.

2. The method according to claim 1, wherein the enteric polymeric film former comprises a polymer containing an acid group.

3. The method according to claim 1, wherein the enteric polymeric film former comprises a methacrylic acid-acrylate copolymer.

4. The method according to claim 1, wherein the enteric polymeric film former comprises a copolymer of methacrylic acid and ethyl acrylate.

5. The method according to claim 1, wherein the enteric polymeric film former is present in a partially neutralized form.

6. The method according to claim 1, wherein the pigment comprises an inorganic pigment, an organic pigments, or mixtures thereof.

7. The method according to claim 6, wherein the pigment comprises a white pigment.

8. The method according to claim 7, wherein the white pigment comprises titanium dioxide.

9. The method according to claim 1, wherein the aqueous pigment dispersion comprises a polyethylene glycol as a plasticizer.

10. The method according to claim 9, wherein the polyethylene glycol has an average molecular weight of from 1500 to 6000 g/mol.

11. The method according to claim 9, wherein the plasticizer comprises a polyethylene glycol-polyvinyl alcohol copolymer.

12. The method according to claim 1, wherein the aqueous pigment dispersion additionally comprises an antisticking agent.

13. The method according to claim 12, wherein the antisticking agent comprises talc.

14. The method according to claim 1, wherein the aqueous pigment dispersion comprises a solids content of from 5 to 40% by weight.

15. The method according to claim 1, wherein the entry air temperature during the spraying process is 20 to 50° C.

16. The method according to claim 1, wherein the exit air humidity during the spraying process is in the range from 20 to 50% relative humidity.

17. The method according to claim 1, wherein the product temperature in the fluidized bed is in the range from 15 to 60° C.

18. The method according to claim 17, wherein the product temperature in the fluidized bed is in the range from 20 to 50° C.

19. The method according to claim 18, wherein the product temperature in the fluidized bed is in the range from 25 to 45° C.

20. The method according to claim 1, wherein the spraying pressure of the aqueous pigment dispersion is in the range from 0.05 to 0.5 MPa.

21. A granule for pharmaceutical applications comprising enteric polymeric film formers obtainable by a method according to claim 1.

22. The granule according to claim 21, having a particle size of from 200 to 600 μm (weighted volume average).

23. The granule according to claim 21, comprising:
(i) 50 to 93% by weight of an enteric polymer;
(ii) 2 to 30% by weight of a pigment;
(iii) 5 to 25% by weight of a plasticizer;
(iv) 0 to 30% by weight of an antisticking agent; and
(v) 0 to 15% by weight of further auxiliaries;
where the quantitative data are based on the total weight of the granules equals 100% by weight.

24. The granule according to claim 23, comprising:
(vi) 70 to 90% by weight of the enteric polymer;
(vii) 2 to 18% by weight of the pigment;
(viii) 7 to 15% by weight of the plasticizer;
(ix) 1 to 20% by weight of the antisticking agent; and
(x) 0 to 5% by weight of the further auxiliaries.

25. The granule according to claim 24, comprising:
(xi) 75 to 80% by weight of the enteric polymer;
(xii) 2 to 8% by weight of the pigment;
(xiii) 10 to 15% by weight of the plasticizer;
(xiv) 7 to 12% by weight of the antisticking agent; and
(xv) 0 to 2% by weight of the further auxiliaries.

26. The granule according to claim 25, comprising 75 to 80% by weight of a partially neutralized copolymer of methacrylic acid ethyl acrylate in the monomer ratio 1:1 as the enteric polymer, 10 to 15% by weight of a polyethylene glycol with molecular weights in the range from 1500 to 6000 daltons as the plasticizer and 7 to 12% by weight of talc as the antisticking agent.

27. A method of producing a pharmaceutical dosage forms, the method comprising using granules with a pharmaceutical to form the pharmaceutical dosage form, wherein the granules are produced by a spraying process in which the enteric polymeric film former is introduced as initial charge in a fluidized bed, and an aqueous pigment suspension which comprises a plasticizer that is solid at 20° C. is sprayed onto the fluidized bed.

28. The method according to claim 27, where a film coating composition comprises at least 80% by weight, based on the solids content, of the granules, which comprise.
(i) 50 to 93% by weight of an enteric polymer;
(ii) 2 to 30% by weight of a pigment;
(iii) 5 to 25% by weight of a plasticizer;
(iv) 0 to 30% by weight of an antisticking agent; and
(v) 0 to 15% by weight of further auxiliaries;
where the quantitative data are based on the total weight of the granules equals 100% by weight.

29. The method according to claim 28, wherein the pigment comprises colored pigments or colored pigment preparations.

30. The method according to claim 28, where the plasticizer comprises a polyethylene glycol.
